(19) United States
(12) Patent Application Publication
(10) Pub. No.: US 2026/0207254 A1
(43) Pub. Date: Jul. 23, 2026

(54) GRAPHICAL DISPLAY FOR AN ELECTROANATOMICAL MAPPING SYSTEM

(71) Applicant: Boston Scientific Scimed, Inc., Maple Grove, MN (US)

(72) Inventor: Meytal Shraga, Brookline, MA (US)

(21) Appl. No.: 19/453,931

(22) Filed: Jan. 20, 2026

Related U.S. Application Data
(60) Provisional application No. 63/746,856, filed on Jan. 17, 2025.

(51) Int. Cl. A61B 18/14; A61B 5/339; A61B 5/367; A61B 17/00; A61B 18/00

(52) U.S. Cl. CPC A61B 18/1492; A61B 5/339; A61B 5/367; A61B 2017/00199; A61B 2018/00351; A61B 2018/00577; A61B 2018/00613; A61B 2018/00875; A61B 2018/1407

(57) ABSTRACT

A method of displaying an indication of proximity of an ablation catheter to tissue, the ablation catheter having a a plurality of splines each having at least one electrode disposed thereon, the method comprising: displaying an icon representing the spline assembly, the icon including: a plurality of spokes extending from a central portion, each spoke corresponding to a respective spline of the plurality of splines and defined by a spoke border, a baseline impedance indictor superimposed at a first location on each spoke; a threshold impedance indicator superimposed at a second location on each spoke, the second location being different than the first location and defined by a user selection of a threshold impedance value; and a calculated impedance indicator disposed within the spoke border, the calculated impedance indicator corresponding to a measured impedance value associated with at least one of the electrodes on the respective spline.

### CROSS REFERENCE TO RELATED APPLICATIONS

This application claims priority to U.S. Provisional Patent Application No. 63/746,856 entitled “GRAPHICAL DISPLAY FOR AN ELECTROANATOMICAL MAPPING SYSTEM,” filed Jan. 17, 2025, which is hereby incorporated by reference in its entirety.

### TECHNICAL FIELD

The present disclosure relates to medical systems and methods for ablating tissue in a patient.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2 is a schematic illustration of a portion of an ablation catheter used in the electrophysiology system of FIG. 1.

FIGS. 3A and 3B illustrate an exemplary portion of a graphical display of the electrophysiology system of FIG. 1.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

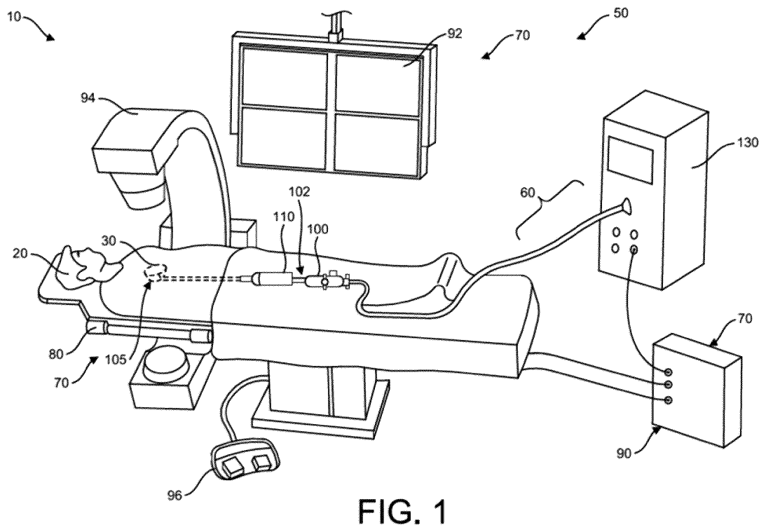

FIG. 1 is a diagram illustrating an exemplary clinical setting 10 for treating a patient 20, and for treating a heart 30 of the patient 20, using an electrophysiology system 50. FIG. 1 illustrates an example clinical setting 10 for treating a patient 20, such as for treating a heart 30 of the patient 20, using an electrophysiology system 50, in accordance with the disclosure. The electrophysiology system 50 includes an ablation catheter system 60 and an electro-anatomical mapping (EAM) system 70. The example ablation catheter system 60 includes an ablation catheter 105, an introducer sheath 110, and an electroporation console 130. Additionally, the ablation catheter system 60 includes various connecting elements, such as cables and tubing, that operably connect the components of the ablation catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The ablation catheter system 60 is configured to deliver ablation electric field energy to targeted tissue in the patient's heart 30 to create cell death in tissue, for example, rendering the tissue incapable of conducting electrical signals. Also, the ablation catheter system 60 is configured to generate electric fields using the ablation catheter 105 to create and present, on the display 92, an electro-anatomical map of the patient's heart to aid a clinician in planning ablation by irreversible electroporation using the ablation catheter 105 prior to delivering ablation electric field energy. In embodiments, the ablation catheter system 60 is configured to generate the electric fields based on characteristics of the ablation catheter 105 and the position of the ablation catheter 105 in the patient 20, such as in the heart 30 of the patient 20. The ablation catheter system 60 is configured to generate graphical representations of the ablation catheter and the electro-anatomical map based on characteristics of the ablation catheter 105 and the position of the ablation catheter 105 in the patient 20, such as in the heart 30 of the patient 20, and the characteristics of the tissue surrounding the catheter 105, such as measured impedances of the tissue. The ablation catheter system 60 can include additional features.

The introducer sheath 110 is operable to provide a delivery conduit through which the ablation catheter 105 can be deployed to the specific target sites within the patient's heart 30. Access to the patient's heart can be obtained through a vessel, such as a peripheral artery or vein often in the groin or possibly in the shoulder or neck. Once access to the vessel is obtained, the ablation catheter 105 can be navigated to within the patient's heart, such as within a chamber of the heart.

In one example, the ablation catheter 105 can be deployed in mapping procedures in cooperation with the EAM system 70 as well as to deliver ablation electric field energy and ablate tissue via irreversible electroporation. The example ablation catheter 105 includes an elongated catheter shaft and distal end region configured to be deployed proximate target tissue, such as within a chamber of the patient's heart or the wall of a pulmonary vein ostium. The shaft can extend from an access point in the patient to the target tissue and generally defines a longitudinal axis of the ablation catheter 105. A proximal end region of the catheter can include a handle having user manipulatable controls for the catheter 105. The distal end region may include a basket, balloon, spline, configured tip, or other electrode deployment mechanism coupled to the shaft. The electrode deployment mechanism includes an electrode assembly, or array, comprising an electrode. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. For instance, the electrode deployment mechanism includes a plurality of flexible support members configured to form a basket, and at least a some of the electrodes are disposed on the flexible support members.

The ablation catheter 105 is configurable in a plurality of states. For example, when the distal end region of the catheter 105 is within a sheath as a catheter assembly, such as to travel to the patient to the chamber of the heart, the electrode deployment mechanism and electrode assembly are in a collapsed state to fit within the sheath. Once the catheter has reached the destination in the chamber of the heart, for example, the sheath is retracted from the distal region of the catheter 105 (or the shaft catheter is extended past the sheath) and the electrode deployment mechanism and electrode assembly can be arranged in an expanded state. The electrode assembly has a collapsed shape when the catheter 105 is in the collapsed state and an expanded shape when the catheter 105 is in the expanded state. In some examples, the electrode assembly has more than two states.

The electroporation console 130 includes a controller, such one or more controllers, processors, or computers, that executes instructions or code, such as processor-executable instructions, out of a non-transitory computer readable medium, such as a memory device, or memory, to cause, such as control or perform, the aspects of the ablation catheter system 60. In one example, the electroporation console 130 is configured to provide an electrical signal, such as a plurality of concurrent or space-apart-time electrical signals, to the electrically connected ablation catheter 105 along lead conductors to the spaced-apart electrodes. The spaced-apart electrodes are configured to generate a selected electrical field proximate the target tissue, based on the electrical signals from the electroporation console 130, such as to effect ablation.

The electroporation console 130 can generate electrical signals and select which electrodes in the electrode array will receive the electrical signals. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes.

In an ablation mode, the console can select electrodes to provide pulsed field ablation (PFA). For example, PFA can be performed with monophasic waveforms and biphasic waveforms. Electric field strengths that are sufficiently high have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

The EAM system 70 is configured to generate the electro-anatomical map for display on the display 92. The EAM system 70 is operable to track the location of the various components of the ablation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the OPAL HDx™ mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the ablation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the OPAL HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the ablation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

In the various embodiments of the disclosure, the EAM system 70, in conjunction with the ablation catheter system 60, can operate to assess the proximity of one or more of the catheter electrodes to the target endocardial tissue, and to provide a visual indication of such proximity to the clinician, as will be explained in greater detail below.

The depiction of the electrophysiology system 50 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

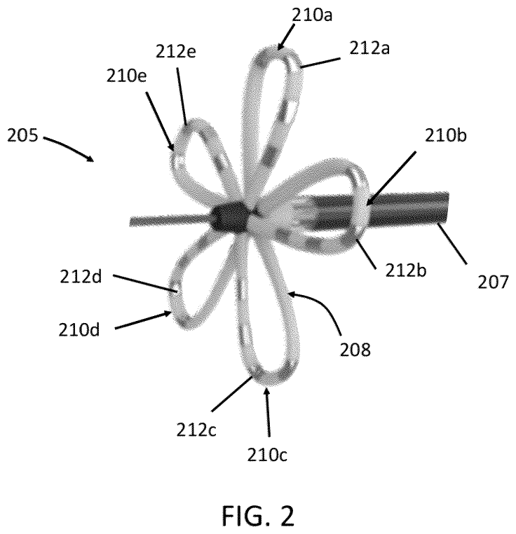

FIG. 2 is a schematic illustration of a portion of an ablation catheter 205 in a deployed state, according to an embodiment of the disclosure. As shown, the ablation catheter 205 includes a shaft 207 and a spline assembly 208 at the end of the shaft 207. As further shown, the spline assembly 207 comprises a plurality of splines 210a, 210b, 210c, 210d and 210e arranged circumferentially relative to the distal end of the shaft. Additionally, at least one spline electrode is disposed on each spline 210a-210e, e.g., spline electrodes 212a, 212b, 212c, 212d and 212e are disposed, respectively, on 210a, 210b, 210c, 210d and 210e. In the illustrated embodiment, each spline has a plurality of electrodes disposed thereon, although the specific number of electrodes on each spline, or in total, can vary and is not critical to the present disclosure. Additionally, the number of individual splines can vary among embodiments. In one embodiment, the ablation catheter 205 may be a FARAWAVE™ pulsed field ablation catheter marketed by Boston Scientific Corporation. In other embodiments, other catheter configurations/designs can be utilized within the scope of the disclosure, e.g., balloon catheters with electrode disposed thereon. It will be further appreciated that the particular embodiment illustrated in FIG. 2 is shown in a fully-deployed configuration of the spline assembly 208. although the specific state of deployment is not critical to the present disclosure.

Referring to FIGS. 1 and 2 collectively, the electrophysiology system 50 includes hardware and software to provide a determination of the one or more electrodes on each spline relative to tissue when in use, and further to provide the user with a visual indication of such proximity on a spline-by-spline basis, e.g., via the display 92. In various embodiments, the aforementioned electrode-tissue proximity can be determined based on a calculated impedance value associated with selected electrode(s) and corresponding tissue. In one embodiment, a current (generated, for example, by the electroporation console 130, see FIG. 1) may be driven at a selected frequency between two or more of the spline electrodes on a given spline 210a, 210b, 210c, 210d or 210e, and a corresponding voltage sensed with respect to one or more of the spline electrodes may be utilized to calculate a local impedance value associated with the driven spline electrodes, which may provide an indication of whether the spline on which the driven spline electrodes is disposed is in proximity to, or in contact with, endocardial tissue. It is emphasized, however, that the particular technique or algorithm utilized in deriving the measured impedance value, or the tissue proximity determination in general, is not critical to the embodiments of the present disclosure.

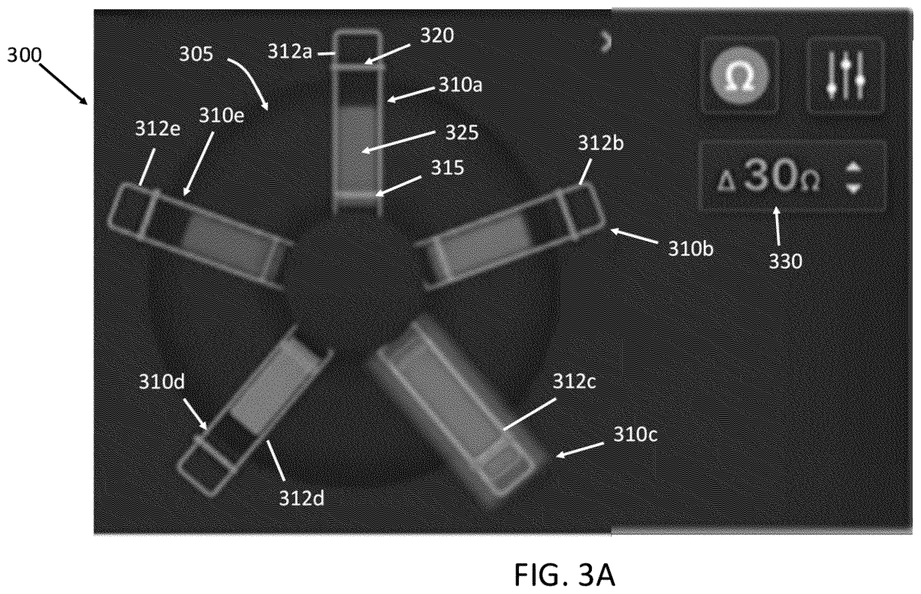

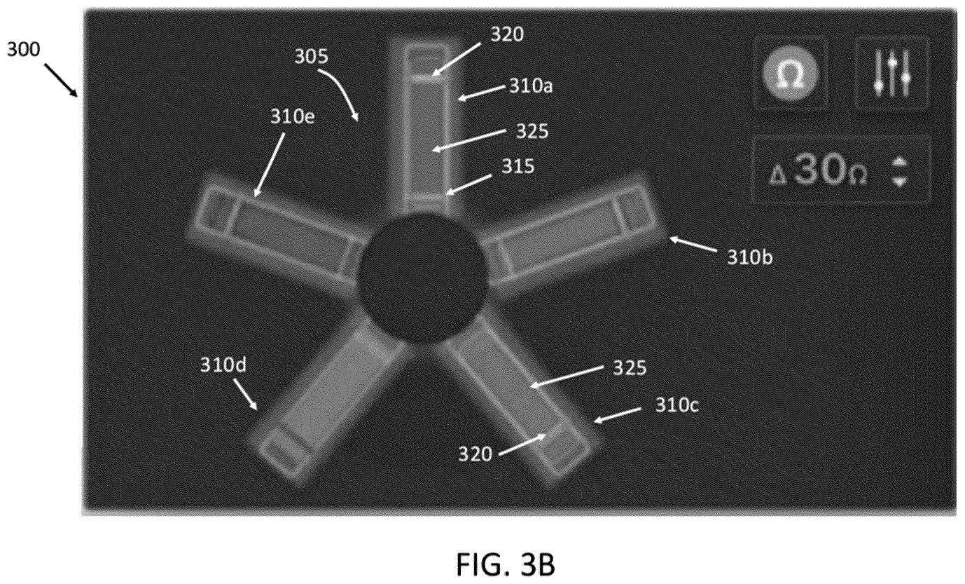

FIGS. 3A and 3B illustrate an exemplary portion of a graphical display 300, which may be incorporated into the display 92 of FIG. 1, to provide the user with an indication of proximity of one or more electrodes on a given spline with tissue. In some cases, the indicated proximity may correspond to actual contact between one or more electrodes on a given spline and the target tissue.

As shown, the graphical display 300 includes an icon 305 providing a graphical schematic representation of the spline assembly 208 (FIG. 2) of the corresponding ablation catheter. As further shown, the icon 5 includes a plurality of spokes arranged circumferentially around and extending radially from a central portion, i.e., spokes 310a, 310b, 310c, 310d and 310e, schematically corresponding to the respective splines 210a, 210b, 210c, 210d and 210e (FIG. 2). Additionally, each spoke 310a, 310b, 310c, 310d and 310e is bounded and defined by a respective spoke border 312a, 312b, 312c, 310d and 312e. In the illustrated embodiment, the circumferential arrangement of the spokes 310a, 310b, 310c, 310d and 310e about the central portion schematically corresponds to the circumferential arrangement of the splines 210a, 210b, 210c, 210d and 210e relative to the shaft 207 (FIG. 2).

As further shown, annotated on each spoke 310a, 310b, 310c, 310d and 310e is a baseline impedance indicator 315, a threshold impedance indicator 320, and a calculated impedance indicator 325. For ease of illustration, the baseline impedance indicator 315, the threshold impedance indicator 320, and the calculated impedance indicator 325 are only labeled with respect to the spoke 310a, although the skilled artisan, based on FIGS. 3A and 3B as a whole, will readily recognize that these features are also shown in connection with the other spokes 310b, 310c, 310d and 310e.

As shown, for each spoke, the baseline impedance indicator 315 is superimposed on the spoke at a first location, and the threshold impedance indicator 320 is superimposed on the spoke at a second location that is different than the first location. In embodiments, the baseline impedance indicator 315 may correspond to a calculated value corresponding to a calculated impedance when the spline assembly is known to be located at a distance away from cardiac tissue, e.g., in the blood pool, and the first location is defined in relation to this calculated baseline impedance. In embodiments, the threshold impedance indicator 320 may correspond to a user-selected value, and accordingly, the graphical display 300 also includes a user-input field 330 whereby the user may, e.g., via a cursor or other input means, select a threshold impedance value. In some embodiments, this threshold impedance value may represent a difference between a calculated impedance value associated with a given spline electrode/spline and the baseline impedance value. Accordingly, in such embodiments, the greater the selected threshold impedance value, the greater the distance will be between the first and second locations.

In embodiments, the calculated impedance indicator 325 on each spoke corresponds to a calculated impedance value associated with at least one spline electrode on the spline corresponding to that spoke. In other words, the length of the calculated impedance indicator 325 on a given spoke may change as that spoke, and at least one spline electrode on the corresponding spline, moves relative to the cardiac tissue. In some embodiments, the association of the calculated impedance value defining the dimensions of the calculated impedance indicator 325 may relate to the difference between the real-time (or close to real-time) calculated impedance at a given position of the spline to the baseline impedance value, which may provide an indication of the proximity of that spline/electrode to the target tissue.

In embodiments, the spoke border 312a-312e of each spoke is assigned a visual appearance that is dependent on the relation of the calculated impedance value to the threshold impedance value. For example, as can be seen in FIG. 3A, the calculated impedance indicator 325 on the spokes 310a, 310b, 310d and 310e terminates between the corresponding baseline and threshold impedance indicators 315, 320, and the corresponding spoke border has a first visual appearance of each such spoke. In contrast, the calculated impedance indicator 325 on spoke 310c extends beyond the threshold impedance indicator 320, i.e., the calculated impedance value associated with at least one electrode on the corresponding spline exceeds the user-defined or preset threshold value, and the spoke border for the spline 310c has a different visual appearance, e.g., highlighting or a halo surrounding it, indicating that the spline (and spline electrode(s) thereon) corresponding to the spoke 310c is in closer proximity, to proximity to the cardiac tissue than the splines/electrodes corresponding to the remaining spokes. In some cases, the user may interpret the relative visual appearances of the respective spokes to assess that the spline/electrode(s) corresponding to spoke 310c are in contact with cardiac tissue, and the other splines are not. As further illustration, in FIG. 3C, the calculated impedance indicator 325 on all each spoke extends beyond the threshold impedance indicator 320, i.e., the calculated impedance value associated with at least one electrode on the spline corresponding to each spoke exceeds the user-defined or preset threshold value, and the spoke border for all spokes is highlighted to indicate that all splines (and spline electrode(s) thereon) are in close proximity and/or in contact with the cardiac tissue.

In some embodiments, the calculated impedance indicator 325 on each spoke (or some other aspect of each spoke) may be assigned a unique visual appearance, e.g., by color, shading, texture, or the like) relative to the other spokes, and which is the same as a visual appearance scheme that is assigned to the graphical representation of the respective electrodes or splines of the catheter itself on the corresponding electroanatomical map. In this way, the icon 305, together with the representation of the catheter spline assembly itself, provides an elegant, easy-to-interpret visual indication of tissue proximity and/or tissue contact on a spline-by-spline basis.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A method of displaying an indication of proximity of an ablation catheter to tissue in an electroanatomical mapping system, the electroanatomical mapping system including a graphical display, the ablation catheter having a spline assembly comprising a plurality of splines, each spline having at least one spline electrode disposed thereon, the method comprising:
displaying, on the graphical display, an icon representing the spline assembly, the icon including:
a plurality of spokes extending from a central portion, each spoke corresponding to a respective spline of the plurality of splines and defined by a spoke border,
a baseline impedance indictor superimposed at a first location on each spoke;
a threshold impedance indicator superimposed at a second location on each spoke, the second location being different than the first location and defined by a user selection of a threshold impedance value; and
a calculated impedance indicator disposed within the spoke border, the calculated impedance indicator corresponding to a measured impedance value associated with at least one of the electrodes on the respective spline.

2. The method of claim 1, wherein the baseline impedance indicator corresponds to a measured calculated value corresponding to a calculated measured impedance when the spline assembly is known to be located at a distance away from cardiac tissue.

3. The method of claim 2, wherein the threshold impedance indicator is a user-selected value.

4. The method of claim 3, wherein each spoke border has a first visual appearance when the calculated impedance value is less than the threshold impedance value, and a second visual appearance when the calculated impedance value is equal or greater than the threshold impedance value.

5. The method of claim 4, wherein the second visual appearance includes a halo disposed about the spoke border.

6. The method of claim 1, wherein the calculated impedance indicator within each spoke is assigned a unique visual appearance relative to the calculated impedance indicator within the other spokes.

7. The method of claim 6, wherein the unique visual appearance is defined by a color.

8. An apparatus for displaying an indication of proximity of an ablation catheter to tissue, the ablation catheter having a spline assembly comprising a plurality of splines, each spline having at least one spline electrode disposed thereon, the apparatus comprising:
a graphical display including an icon representing the spline assembly, the icon including:
a plurality of spokes extending from a central portion, each spoke corresponding to a respective spline of the plurality of splines and defined by a spoke border;
a baseline impedance indictor superimposed at a first location on each spoke;
a threshold impedance indicator superimposed at a second location on each spoke, the second location being different than the first location and defined by a user selection of a threshold impedance value; and
a calculated impedance indicator disposed within the spoke border, the measured impedance indicator corresponding to a calculated impedance value associated with at least one of the electrodes on the respective spline.

9. The apparatus of claim 8, wherein the baseline impedance indicator corresponds to a measured calculated value corresponding to a calculated measured impedance when the spline assembly is known to be located at a distance away from cardiac tissue.

10. The apparatus of claim 9, wherein the threshold impedance indicator is a user-selected value.

11. The apparatus of claim 8, wherein each spoke border has a first visual appearance when the calculated impedance value is less than the threshold impedance value, and a second visual appearance when the calculated impedance value is equal or greater than the threshold impedance value.

12. The apparatus of claim 11, wherein the second visual appearance includes a halo disposed about the spoke border.

13. The apparatus of claim 8, wherein the calculated impedance indicator within each spoke is assigned a unique visual appearance relative to the measured impedance indicator within the other spokes.

14. The apparatus of claim 13, wherein the unique visual appearance is defined by a color.

15. An electrophysiology system comprising:
an ablation catheter having a catheter shaft and a spline assembly disposed at a distal end of the catheter shaft, the spline assembly comprising a plurality of splines each having one or more spline electrodes disposed thereon;
an electroporation console coupled to the ablation catheter and configured to deliver electrical pulses to the one or more spline electrodes; and
an electroanatomical mapping system coupled to the ablation catheter and including a graphical display, the graphical display including an icon representing the spline assembly, the icon including:
a plurality of spokes extending from a central portion, each spoke corresponding to a respective spline of the plurality of splines and defined by a spoke border;
a baseline impedance indictor superimposed at a first location on each spoke;
a threshold impedance indicator superimposed at a second location on each spoke, the second location being different than the first location and defined by a user selection of a threshold impedance value; and
a calculated impedance indicator disposed within the spoke border, the measured impedance indicator corresponding to a calculated impedance value associated with at least one of the electrodes on the respective spline.

16. The electrophysiology system of claim 15, wherein the baseline impedance indicator corresponds to a measured calculated value corresponding to a calculated measured impedance when the spline assembly is known to be located at a distance away from cardiac tissue.

17. The electrophysiology system of claim 16, wherein the threshold impedance indicator is a user-selected value.

18. The electrophysiology system of claim 15, wherein each spoke border has a first visual appearance when the calculated impedance value is less than the threshold impedance value, and a second visual appearance when the calculated impedance value is equal or greater than the threshold impedance value.

19. The electrophysiology system of claim 15, wherein the calculated impedance indicator within each spoke is assigned a unique visual appearance relative to the measured impedance indicator within the other spokes.

20. The electrophysiology system of claim 19, wherein the unique visual appearance is defined by a color.
